(19) United States
(12) Patent Application Publication
(10) Pub. No.: US 2008/0138309 A1
(43) Pub. Date: Jun. 12, 2008

(54) Use Of Aminodithiol As A Reducing Agent For Hair Perming

(76) Inventors: Gerald Malle, Villiers S/Morin (FR); Christian Blaise, Saint Mande (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/795,608

(22) PCT Filed: Jan. 19, 2006
(86) PCT No.: PCT/EP06/01137

§ 371 Date: Nov. 8, 2007

Related U.S. Application Data
(60) Provisional application No. 60/651,659, filed on Feb. 11, 2005.

(30) Foreign Application Priority Data
Jan. 20, 2005 (FR) 0550178

(51) Int. Cl. A61K 8/46; C07C 321/04

(52) U.S. Cl. 424/70.51; 562/556; 564/154

(57) ABSTRACT

The present invention relates to the use of aminodithiols as reducing agents for hair perming. It further relates to a hair perming method using these aminodithiols.

The present invention relates to the use of aminodithiols as reducing agents for hair perming. It further relates to a hair perming method using these aminodithiols.

The most common technique for perming hair consists in a first step in opening the disulphide bonds of the keratin (cystine) with a composition containing a reducing agent, and then in a second step, preferably after having rinsed the hair and after having stretched it on rollers or the like or shaped or smoothed it by other means, in re-forming said disulphide bonds by applying an oxidizing composition, also called a fixative, so as to give the hair the desired shape. This technique makes it possible equally well to wave, uncurl, straighten or smooth the hair.

The reducing compositions generally used for the first step of a perming operation contain sulphites, bisulphites or, preferably, thiols as reducing agent. Among the latter, those commonly used are thioglycolic acid, cysteamine, thiolactic acid, cysteine and glycerol monothioglycolate. Thioglycolic acid is particularly effective for reducing the disulphide bonds of the keratin at alkaline pH, especially in the form of ammonium thioglycolate, and is the product most widely used in perming. However, it was found that thioglycolic acid had to be used in a sufficiently basic medium (in practice at pH≧8.5) if it was desired to obtain curling of satisfactory intensity. Apart from the disadvantage of releasing an unpleasant odour requiring the use of more or less effective perfumes to mask the odours, the combination thioglycolic acid+alkaline pH causes degradations of the hair fibre.

Sulphites or bisulphites were used before thiols in general and thioglycolic acid in particular. In contrast to thiols, they are used at an acidic pH generally of between 4 and 6. However, the degree of curling obtained is very inferior and far from satisfactory.

Cysteine produces a much weaker odour than thioglycolic acid, but the degree of curling obtained is also very inferior and far from satisfactory. Furthermore, cysteine requires the use of a very alkaline pH.

Glycerol monothioglycolate is also very malodorous. On the other hand, it is used at a pH close to neutrality, but its performance characteristics are appreciably inferior to those of thioglycolic acid.

Cysteamine can be used over a wider pH range. Its efficacy is similar to that of thioglycolic acid, but it also causes substantial degradations of the hair fibre.

Various studies have been conducted with a view to overcoming the disadvantages of these reducing agents, and to this end the use of novel reducing compounds or systems has been proposed. However, very few dithiols have been proposed. In the past, 2 dithiols in particular were widely studied, namely dithiothreitol, DTT, and 2,5-dimercaptoadipic acid, but they were never developed for hair perming, particularly because of the appalling odour of DTT and the insufficient activity of dimercaptoadipic acid. More recently, it has already been proposed in patent application EP-A-0721772 to use 2,3-dimercaptosuccinic acid, which has proved less effective than thioglycolic acid. It has also been proposed in U.S. Pat. No. 5,350,572 to use polyoxyethylene glycol dimercaptoalkyl esters. Although these compounds possess a certain efficacy, they do not keep satisfactorily over time.

After various studies, the Applicant has now discovered, surprisingly and unexpectedly, a novel family of aminodithiols that produces curling of satisfactory intensity and hold, said family also causing less degradation of the keratin fibres.

The present invention relates to the use of at least one aminodithiol of general formula (I), in a reducing composition for the hair, as reducing agent for the first step of an operation for the perming of keratin fibres, especially hair:

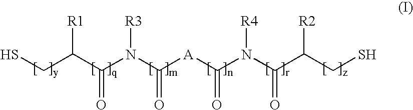

in which:

R₁ and R₂ independently of one another are H, COOR₅ or CONR₆R₇, where R₅=H or linear or branched C₁-C₅ alkyl optionally substituted by 1 or 2 OH radicals, and R₆, R₇=H or linear or branched C₁-C₅ alkyl optionally substituted by 1 or 2 radicals selected from hydroxyl, methylamino, ethylamino and dimethylamino,

R₃ and R₄ independently of one another are H or COOH,

y and z independently of one another are equal to 1 or 2,

A=nothing (0), (CH₂)ₓ, where x ranges from 0 to 6, CH₂—CHOH—CH₂, CH₂—CHOH—CHOH—CH₂, HOOC—CH═CH—COOH or CH₂—CH(NR₈R₉)—(CH₂)ₚ, where p=1 or 2 and R₈, R₉=H, CH₃ or COCH₃,

m, n, q, r=0 or 1 with the following conditions:

(i) if m=n=1, q=r=0

(ii) if m=1 and n=0 and conversely if m=0 and n=1, q and r cannot simultaneously be equal to 1

(iii) if m=n=0, A cannot be nothing

(iv) m, n, q and r can simultaneously be equal to 0 if A is other than nothing,

and their physiologically acceptable organic and inorganic salts.

“Perming” is understood as meaning the permanent curling, the uncurling or the straightening of the hair.

Yet another subject of the invention is a hair perming method that uses a reducing composition comprising at least one compound of formula (I).

The compounds of formula (I) are generally prepared by the procedures described in the following references:
Nuclear Medicine and Biology 1998, 25(2), 135-140
Proceedings of the 7th International Symposium, Dresden, Germany, Jun. 18-22, 2000, 400-403
Tetrahedron Letters 2001, 42(44), 7847-7850
Journal of Medicinal Chemistry 2001, 44(3), 298-301
Journal of Medicinal Chemistry 1965, 8(1), 29-33
Nuclear Science Journal 2000, 37(3), 188-196
Journal of Labelled Compounds and Radiopharmaceuticals 1999, 42(6), 553-565
Nuclear Medicine and Biology 1998, 25(6), 569-575
Applied Radiation and Isotopes 1997, 48(8), 1103-1111
Synthesis and Reactivity in Inorganic and Metal-Organic Chemistry 1997, 27(8), 1167-1182
Inorganic Chemistry 1984, 23(23), 3793-7
Annali di Chimica 1960, 50, 690-7
Acta Bioguimica Clinica Latinoamericana 1993, 27(2), 233-41
Patent application EP635276
Patent application GB799057
Patent application WO 2002053624
Patent application WO 9005733
U.S. Pat. No. 5,986,074
U.S. Pat. No. 5,688,485
U.S. Pat. No. 5,279,811
U.S. Pat. No. 2,810,753

The preferred compounds below may be mentioned in particular among the compounds of general formula (I):
2,2′-oxybis[N-(2-mercaptoethyl)acetamide]
N,N′-(1,2-dioxo-1,2-ethanediyl)bis(L-cysteine dimethyl ester)
N,N′-(1,3-dioxo-1,3-propanediyl)bis(L-cysteine dimethyl ester)
N,N′-(1,4-dioxo-1,4-butanediyl)bis(L-cysteine dimethyl ester)
N,N′-(1,2-dioxo-1,2-ethanediyl)-bis-L-cysteine
N,N′-(1,4-dioxo-1,4-butanediyl)-bis-L-cysteine
N,N′-(1,5-dioxo-1,5-pentanediyl)bis(L-cysteine dimethyl ester)
N,N′-(1,6-dioxo-1,6-hexanediyl)bis(L-cysteine dimethyl ester)
N,N′-(1,7-dioxo-1,7-heptanediyl)bis(L-cysteine dimethyl ester)
N,N′-(1,8-dioxo-1,8-octanediyl)bis(L-cysteine dimethyl ester)
N,N′-(1,2-ethanediyl)bis(L-cysteine dibutyl ester)
N,N′-(1,2-ethanediyl)bis(L-cysteine dimethyl ester)
N,N′-(1,2-ethanediyl)bis(L-cysteine dipropyl ester)
N,N′-(1,2-ethanediyl)-bis-L-cysteine
N,N′-(1,2-ethanediyl)bis(L-cysteine diethyl ester)
N,N′-(1,2-ethanediyl)bis(DL-cysteine diethyl ester)
N,N′-(1,2-ethanediyl)-bis-L-homocysteine
N,N′-(1,3-propanediyl)-bis-L-cysteine
N,N′-(1,3-propanediyl)-bis-L-homocysteine
N,N′-1,3-(2-hydroxypropanediyl)-bis-L-cysteine
N,N′-1,3-(2-hydroxypropanediyl)-bis-L-homocysteine
N,N′-carbonyl-bis(L-cysteine)
N-[2-[[(1R)-1-carboxy-2-mercaptoethyl]amino]ethyl]-L-cysteine 1-ethyl ester
N-[2-[(2-mercaptoethyl)amino]ethyl]-L-cysteine 1-ethyl ester
N-[2-[(2-mercaptoethyl)amino]ethyl]-L-cysteine
N-(2-mercaptoethyl)-2-[(2-mercaptoethyl)amino]-acetamide
N-[(2-mercaptoethyl)glycyl]-L-cysteine
N-[(2-mercaptoethyl)glycyl]-L-cysteine ethyl ester
N-(1-carboxy-2-mercaptoethyl)-3-[(1-carboxy-2-mercaptoethyl)amino]aspartic acid
2,2′-(ethylenediimino) diethanethiol dihydrochloride
3,7-diaza-1,9-nonanedithiol
3,6-diaza-1,8-octanedithiol
N,N′-bis(2-mercaptoethyl)ethanediamide
N,N′-bis(2-mercaptoethyl)butanediamide
N,N′-bis(2-mercaptoethyl)hexanediamide
1,4-bis[(2-mercaptoethyl)amino]-2,3-butanediol
N,N′-bis(mercaptoethyl)urea
1,2-ethanediylbis[(2-mercaptoethyl)carbamic] acid
2-[(1-carboxy-2-mercaptoethylaminooxalyl)amino]-3-mercaptopropionic acid
methyl 3-mercapto-2-[(2-mercapto-1-methoxycarbonylethylaminooxalyl)amino]propionate
ethyl 3-mercapto-2-[(2-mercapto-1-ethoxycarbonylethylaminooxalyl)amino]propionate
N,N′-bis[2-(methylamino)-2-oxo-1-(mercaptomethyl)ethyl]ethanediamide
N,N′-bis[2-(ethylamino)-2-oxo-1-(mercaptomethyl)ethyl]ethanediamide
N,N′-bis[2-(dimethylamino)-2-oxo-1-(mercaptomethyl)ethyl]ethanediamide
N,N′-bis[2-[(2-hydroxypropyl)amino]-2-oxo-1-(mercaptomethyl)ethyl]ethanediamide
N,N′-bis[2-[(2-hydroxyethyl)amino]-2-oxo-1-(mercaptomethyl)ethyl]ethanediamide
N,N′-bis[2-{[2-(dimethylamino)ethyl]amino}-2-oxo-1-(mercaptomethyl)ethyl]ethanediamide
N,N′-bis[2-{[3-(dimethylamino)propyl]amino}-2-oxo-1-(mercaptomethyl)ethyl]ethanediamide-1
2-({3-[(1-carboxy-2-mercaptoethyl)amino]-3-oxopropanoxyl}amino)-3-mercaptopropionic acid
methyl 2-[3-{[2-methoxy-2-oxo-1-(mercaptomethyl)ethyl]amino}-(3-oxopropanoxyl)amino]-3-mercaptopropanoate
ethyl 2-[3-{[2-ethoxy-2-oxo-1-(mercaptomethyl)ethyl]-amino}-(3-oxopropanoxyl)amino]-3-mercaptopropanoate
N,N′-bis[2-(methylamino)-2-oxo-1-(mercaptomethyl)ethyl]malonamide
N,N′-bis[2-(ethylamino)-2-oxo-1-(mercaptomethyl)ethyl]malonamide
N,N′-bis[2-(2-hydroxypropylamino)-2-oxo-1-(mercaptomethyl)ethyl]malonamide
N,N′-bis[2-(2-hydroxyethylamino)-2-oxo-1-(mercaptomethyl)ethyl]malonamide
N,N′-bis[2-(dimethylamino)-2-oxo-1-(mercaptomethyl)ethyl]malonamide
N,N′-bis[2-{[2-(dimethylamino)ethyl]amino}-2-oxo-1-(mercaptomethyl)ethyl]malonamide
N,N′-bis[2-{[3-(dimethylamino)propyl]amino}-2-oxo-1-(mercaptomethyl)ethyl]malonamide
2-({[(1-carboxy-2-mercaptoethyl)amino]acetyl}amino)-3-mercaptopropionic acid
methyl 2-[({[2-methoxy-2-oxo-1-(mercaptomethyl)ethyl]amino}acetyl)amino]-3-mercaptopropanoate
ethyl 2-[({[2-ethoxy-2-oxo-1-(mercaptomethyl)ethyl]amino}acetyl)amino]-3-mercaptopropanoate
N-methyl-2-[({[2-methylamino-2-oxo-1-(mercaptomethyl)ethyl]amino}acetyl)amino]-3-mercaptomethylpropanamide
N-methyl-2-[({[2-hydroxypropylamino-2-oxo-1-(mercaptomethyl)ethyl]amino}acetyl)amino]-3-mercaptomethylpropanamide
N-methyl-2-[({[2-hydroxyethylamino-2-oxo-1-(mercaptomethyl)ethyl]amino}acetyl)amino]-3-mercaptomethylpropanamide
2-({[2-dimethylamino-2-oxo-1-(mercaptomethyl)ethyl]amino}acetyl)amino-N,N-dimethyl-3-mercaptopropanamide
2-({[2-diethylamino-2-oxo-1-(mercaptomethyl)ethyl]amino}acetyl)amino-N,N-dimethyl-3-mercaptopropanamide
N-[2-(dimethylamino)ethyl]-2-methyl-6,9-dioxo-7,12-bis(mercaptomethyl)-2,5,8,11-tetraazamidecan-13-amide
N-[3-(dimethylamino)propyl]-2-methyl-6,9-dioxo-7,12-bis(mercaptomethyl)-2,5,8,11-tetraazamidecan-13-amide
N,N′-1,2-ethanediylbis(3-mercaptopropanamide)
N,N′-1,3-propanediylbis(3-mercaptopropanamide)
N,N′-1,3-(2-hydroxypropanediyl)bis(3-mercaptopropanamide)
N,N′-1,4-butanediylbis(3-mercaptopropanamide)
N,N′-1,5-pentanediylbis(3-mercaptopropanamide)
N,N′-1,6-hexanediylbis(3-mercaptopropanamide)
N,N′-1,2-ethanediylbis(4-mercaptobutanamide)
N,N′-1,3-propanediylbis(4-mercaptobutanamide)
N,N′-1,3-(2-hydroxypropanediyl)bis(4-mercaptobutanamide)
N,N′-1,4-butanediylbis(4-mercaptobutanamide)
N-[2-(2-carboxy-3-mercaptopropionylamino)ethyl]-2-mercaptomethylmalonamic acid
methyl N-[2-(2-methoxycarbonyl-3-mercaptopropionylamino)ethyl]-2-mercaptomethylmalonamate
ethyl N-[2-(2-ethoxycarbonyl-3-mercaptopropionylamino)ethyl]-2-mercaptomethylmalonamate
2-[2-(2-carbamoyl-3-mercaptopropionylamino)ethylcarbamoyl]-3-mercaptopropionamide
3-mercapto-N-[2-(3-mercapto-2-methylcarbamoylpropionylamino)ethyl]-2-methylcarbamoylpropionamide
3-mercapto-N-[2-(3-mercapto-2-ethylcarbamoylpropionylamino)ethyl]-2-ethylcarbamoylpropionamide
2-dimethylcarbamoyl-N-[2-(2-dimethylcarbamoyl-3-mercaptopropionylamino)ethyl]-3-mercaptopropionamide
N-[3-(2-carboxy-3-mercaptopropionylamino)propyl]-2-mercaptomethylmalonamic acid
methyl N-[3-(2-methoxycarbonyl-3-mercaptopropionylamino)propyl]-2-mercaptomethylmalonamate
ethyl N-[3-(2-ethoxycarbonyl-3-mercaptopropionylamino)propyl]-2-mercaptomethylmalonamate
2-[3-(2-carbamoyl-3-mercaptopropionylamino)propylcarbamoyl]-3-mercaptopropionamide
3-mercapto-N-[3-(3-mercapto-2-methylcarbamoylpropionylamino)propyl]-2-methylcarbamoylpropionamide
3-mercapto-N-[3-(3-mercapto-2-ethylcarbamoylpropionylamino)propyl]-2-ethylcarbamoylpropionamide
2-dimethylcarbamoyl-N-[3-(2-dimethylcarbamoyl-3-mercaptopropionylamino)propyl]-3-mercaptopropionamide
N-[3-(2-carboxy-3-mercaptopropionylamino)-2-hydroxypropyl]-2-mercaptomethylmalonamic acid
methyl N-[3-(2-methoxycarbonyl-3-mercaptopropionylamino)-2-hydroxypropyl]-2-mercaptomethylmalonamate
ethyl N-[3-(2-ethoxycarbonyl-3-mercaptopropionylamino)-2-hydroxypropyl]-2-mercaptomethylmalonamate
2-[3-(2-carbamoyl-3-mercaptopropionylamino)-2-hydroxypropylcarbamoyl]-3-mercaptopropionamide
3-mercapto-N-[3-(3-mercapto-2-methylcarbamoylpropionylamino)-2-hydroxypropyl]-2-methylcarbamoylpropionamide
3-mercapto-N-[3-(3-mercapto-2-ethylcarbamoylpropionylamino)-2-hydroxypropyl]-2-ethylcarbamoylpropionamide
2-dimethylcarbamoyl-N-[3-(2-dimethylcarbamoyl-3-mercaptopropionylamino)-2-hydroxypropyl]-3-mercaptopropionamide.

The following compounds are very particularly preferred:
2,2′-oxybis[N-(2-mercaptoethyl)acetamide]
N,N′-(1,2-dioxo-1,2-ethanediyl)bis(L-cysteine dimethyl ester)
N,N′-(1,4-dioxo-1,4-butanediyl)bis(L-cysteine dimethyl ester)
N,N′-(1,4-dioxo-1,4-butanediyl)-bis-L-cysteine
N,N′-(1,2-ethanediyl)-bis-L-cysteine
N,N′-(1,2-ethanediyl)-bis-L-homocysteine
N,N′-(1,3-propanediyl)-bis-L-cysteine
N,N′-(1,3-propanediyl)-bis-L-homocysteine
N,N′-1,3-(2-hydroxypropanediyl)-bis-L-cysteine
N,N′-1,3-(2-hydroxypropanediyl)-bis-L-homocysteine
N,N′-carbonylbis(L-cysteine)
N-[2-[[(1R)-1-carboxy-2-mercaptoethyl]amino]ethyl]-L-cysteine 1-ethyl ester
N-[2-[(2-mercaptoethyl)amino]ethyl]-L-cysteine 1-ethyl ester
N-[2-[(2-mercaptoethyl)amino]ethyl]-L-cysteine
N-(2-mercaptoethyl)-2-[(2-mercaptoethyl)amino]-acetamide
N-[(2-mercaptoethyl)glycyl]-L-cysteine
N-(1-carboxy-2-mercaptoethyl)-3-[(1-carboxy-2-mercaptoethyl)amino]aspartic acid
2,2′-(ethylenediimino)diethanethiol dihydrochloride
3,6-diaza-1,8-octanedithiol
N,N′-bis(2-mercaptoethyl)ethanediamide
N,N′-bis(2-mercaptoethyl)butanediamide
N,N′-bis(2-mercaptoethyl)hexanediamide
1,4-bis[(2-mercaptoethyl)amino]-2,3-butanediol
N,N′-bis(mercaptoethyl)urea
1,2-ethanediylbis[(2-mercaptoethyl)carbamic] acid
N,N′-1,2-ethanediylbis(3-mercaptopropanamide)
N,N′-1,3-propanediylbis(3-mercaptopropanamide)
N,N′-1,3-(2-hydroxypropanediyl)bis(3-mercaptopropanamide)
N,N′-1,4-butanediylbis(3-mercaptopropanamide)
N,N′-1,5-pentanediylbis(3-mercaptopropanamide)
N,N′-1,2-ethanediylbis(4-mercaptobutanamide)
N,N′-1,3-propanediylbis(4-mercaptobutanamide)
N,N′-1,3-(2-hydroxypropanediyl)bis(4-mercaptobutanamide)
N,N′-1,4-butanediylbis(4-mercaptobutanamide)
N-[2-(2-carboxy-3-mercaptopropionylamino)ethyl]-2-mercaptomethylmalonamic acid
methyl N-[2-(2-methoxycarbonyl-3-mercaptopropionylamino)ethyl]-2-mercaptomethylmalonamate
ethyl N-[2-(2-ethoxycarbonyl-3-mercaptopropionylamino)ethyl]-2-mercaptomethylmalonamate
2-[2-(2-carbamoyl-3-mercaptopropionylamino)ethylcarbamoyl]-3-mercaptopropionamide
3-mercapto-N-[2-(3-mercapto-2-methylcarbamoylpropionylamino)ethyl]-2-methylcarbamoylpropionamide
3-mercapto-N-[2-(3-mercapto-2-ethylcarbamoylpropionylamino)ethyl]-2-ethylcarbamoylpropionamide
2-dimethylcarbamoyl-N-[2-(2-dimethylcarbamoyl-3-mercaptopropionylamino)ethyl]-3-mercaptopropionamide
N-[3-(2-carboxy-3-mercaptopropionylamino)propyl]-2-mercaptomethylmalonamic acid
methyl N-[3-(2-methoxycarbonyl-3-mercaptopropionylamino)propyl]-2-mercaptomethylmalonamate
ethyl N-[3-(2-ethoxycarbonyl-3-mercaptopropionylamino)propyl]-2-mercaptomethylmalonamate
2-[3-(2-carbamoyl-3-mercaptopropionylamino)propylcarbamoyl]-3-mercaptopropionamide
3-mercapto-N-[3-(3-mercapto-2-methylcarbamoylpropionylamino)propyl]-2-methylcarbamoylpropionamide
3-mercapto-N-[3-(3-mercapto-2-ethylcarbamoylpropionylamino)propyl]-2-ethylcarbamoylpropionamide
2-dimethylcarbamoyl-N-[3-(2-dimethylcarbamoyl-3-mercaptopropionylamino)propyl]-3-mercaptopropionamide
N-[3-(2-carboxy-3-mercaptopropionylamino)-2-hydroxypropyl]-2-mercaptomethylmalonamic acid
methyl N-[3-(2-methoxycarbonyl-3-mercaptopropionylamino)-2-hydroxypropyl]-2-mercaptomethylmalonamate
ethyl N-[3-(2-ethoxycarbonyl-3-mercaptopropionylamino)-2-hydroxypropyl]-2-mercaptomethylmalonamate
2-[3-(2-carbamoyl-3-mercaptopropionylamino)-2-hydroxypropylcarbamoyl]-3-mercaptopropionamide
3-mercapto-N-[3-(3-mercapto-2-methylcarbamoylpropionylamino)-2-hydroxypropyl]-2-methylcarbamoylpropionamide
3-mercapto-N-[3-(3-mercapto-2-ethylcarbamoylpropionylamino)-2-hydroxypropyl]-2-ethylcarbamoylpropionamide
2-dimethylcarbamoyl-N-[3-(2-dimethylcarbamoyl-3-mercaptopropionylamino)-2-hydroxypropyl]-3-mercaptopropionamide.

The hair perming method according to the invention comprises the application of a reducing composition comprising a compound of formula (I) as reducing agent. The hair is shaped using mechanical means well known to those skilled in the art, such as rollers, the reducing composition being applied before or after the hair shaping means, and a fixing composition being applied after the reducing composition, with or without an intermediate or subsequent step for rinsing or applying an intermediate composition.

According to the present invention, hair perming preferably consists, in a first step, in reducing the disulphide bonds of the keratin by applying a reducing composition as defined above for about 5 to 60 min and then, in a second step, in re-forming said bonds by applying an oxidizing composition or optionally by allowing the atmospheric oxygen to take effect.

Preferably, a reducing composition as defined above is applied to wet hair which has first been wound onto rollers of 2 to 30 mm in diameter, it optionally being possible for the composition to be applied as the hair is being rolled; the reducing composition is subsequently allowed to take effect for a period of 5 to 60 minutes, preferably of 15 to 45 minutes, and then rinsed off with copious amounts of water; an oxidizing composition is then applied to the rolled hair for an exposure time of 2 to 10 minutes in order to re-form the disulphide bonds of the keratin. After the rollers have been removed, the hair is rinsed with copious amounts of water.

Another advantageous possibility, after application of the reducing composition, is to subject the hair to a heat treatment by heating to a temperature of between 30 and 60° C., especially for all or part of the exposure time. In practice this operation can be carried out by means of a hood, a hair dryer, an infrared radiator or other conventional heating apparatus.

It is also possible to use heating tongs at a temperature of between 60 and 220° C., preferably of between 120 and 200° C., as both heating means and hair shaping means.

The oxidizing composition is of the commonly used type and contains e.g. hydrogen peroxide, an alkali metal bromate, a per salt, a polythionate or an alkali metal bromate/per salt mixture as oxidizing agent. The hydrogen peroxide concentration can vary from 1 to 20 volumes, preferably from 1 to 10 volumes, the alkali metal bromate concentration can vary from 2 to 12% and the per salt concentration can vary from 0.1 to 15% by weight, based on the total weight of the oxidizing composition. The pH of the oxidizing composition is generally between 2 and 10. This oxidation can be effected immediately or deferred.

The hair shaping according to the invention can also consist of a hair uncurling or straightening method in which a reducing composition according to the invention is applied to the hair, after which the hair is subjected to mechanical shaping in order to fix it in its new shape, especially by means of a hair smoothing operation with a wide-toothed comb, the back of a comb or the hand. After an exposure time of 5 to 60 minutes, particularly of 15 to 45 minutes, the hair is smoothed again and then rinsed carefully, an oxidizing or fixing composition as defined above is applied and allowed to take effect for about 2 to 10 minutes, and then the hair is rinsed with copious amounts of water.

Heating tongs at a temperature of between 60 and 220° C., preferably of between 120 and 200° C., can be used as both heating means and hair smoothing means.

In the reducing compositions for the hair according to the invention, the reducing agent of general formula (I) is advantageously present, in a cosmetically acceptable medium, at a concentration of between 0.05 and 35% by weight, preferably of between 1 and 25% by weight, based on the total weight of the reducing composition.

The pH of the reducing composition for the hair is preferably between 4 and 11, more particularly between 6 and 10. In general it is obtained with an alkaline agent, e.g. aqueous ammonia, monoethanolamine, diethanolamine, triethanolamine, propane-1,3-diamine, an alkali metal or ammonium carbonate or bicarbonate, an organic carbonate such as guanidine carbonate, or an alkali metal hydroxide, or with an acidifying agent, e.g. hydrochloric acid, acetic acid, lactic acid, oxalic acid or boric acid, or else with customary buffers such as borate, phosphate, TRIS or other buffers.

The reducing composition for the hair can also contain other known reducing agents, e.g. thioglycolic acid or thiolactic acid and their ester and amide derivatives, especially glycerol monothioglycolate, cysteamine and its C₁-C₄ acylated derivatives such as N-acetylcysteamine or N-propionylcysteamine, cysteine, N-acetylcysteine, thiomalic acid, pantetheine, 2,3-dimercaptosuccinic acid, alkali metal or alkaline earth metal sulphites or bisulphites, the N-(mercaptoalkyl)co-hydroxyalkylamides described in patent application EP-A-354 835, the N-monoalkyl- or N,N-dialkylmercapto-4-butyramides described in patent application EP-A-368 763, the aminomercaptoalkylamides described in patent application EP-A-432 000, the N-(mercaptoalkyl)succinamic acid and N-(mercaptoalkyl)succinimide derivatives described in patent application EP-A-465 342, the alkylaminomercaptoalkylamides described in patent application EP-A-514 282, the azeotropic mixture of 2-hydroxypropyl thioglycolate and 2-hydroxy-1-methylethyl thioglycolate described in patent application FR-A-2 679 448, the mercaptoalkylaminoamides described in patent application FR-A-2 692 481, the N-mercaptoalkylalkanediamides described in patent application EP-A-653 202, and the formamidinesulphinic acid derivatives described in patent application PCT/US01/43124 filed by the Applicant.

In one preferred embodiment, the reducing composition for the hair also contains a surfactant of non-ionic, anionic, cationic or amphoteric type, among which the following may be mentioned: alkylsulphates, alkylbenzenesulphates, alkylethersulphates, alkylsulphonates, quaternary ammonium salts, alkylbetaines, ethoxylated alkylphenols, fatty acid alkanolamides, ethoxylated fatty acid esters and other non-ionic surfactants of the hydroxypropyl ether type.

If the reducing composition for the hair contains at least one surfactant, this is generally present at a maximum concentration of 30% by weight, preferably of between 0.5 and 10% by weight, based on the total weight of the reducing composition.

In order to improve the cosmetic properties of the hair or to decrease or avoid its degradation, the reducing composition can also contain a treating agent of a cationic, anionic, non-ionic or amphoteric nature.

The treating agents described in French patents no. 2.598.613 and no. 2.470.596 may be mentioned especially among those which are particularly preferred. Other treating agents which can be used are volatile or non-volatile linear or cyclic silicones and mixtures thereof, polydimethylsiloxanes, quarternized polyorganosiloxanes such as those described in French patent application no. 2.535.730, polyorganosiloxanes with aminoalkyl groups modified by alkoxycarbonylalkyl groups, such as those described in U.S. Pat. No. 4,749,732, polyorganosiloxanes such as the polydimethylsiloxane/polyoxyalkyl copolymer of the dimethicone copolyol type, a polydimethylsiloxane with stearyloxy end groups (stearyloxydimethicone), a polydimethylsiloxane/dialkylammonium acetate copolymer or a polydimethylsiloxane/polyalkylbetaine copolymer as described in British patent no. 2.197.352, polysiloxanes organo-modified by mercapto or mercaptoalkyl groups, such as those described in French patent no. 1.530.369 and European patent application no. 295.780, and silanes such as stearyloxytrimethylsilane.

The reducing composition can also contain other treating ingredients such as cationic polymers like those used in the compositions of French patents no. 79.32078 (2.472.382) and 80.26421 (2.495.931), or cationic polymers of the ionene type, such as those used in the compositions of Luxemburg patent no. 83703, basic amino acids (such as lysine, arginine) or acidic amino acids (such as glutamic acid, aspartic acid), peptides and derivatives thereof, protein hydrolysates, waxes, swelling and penetrating agents or agents for enhancing the efficacy of the reducing agent, such as the SiO₂/PDMS (polydimethylsiloxane) mixture, dimethylisosorbitol, urea and derivatives thereof, pyrrolidone, N-alkylpyrrolidones, thiamorpholinone, alkylene glycol or dialkylene glycol alkyl ethers, e.g. propylene glycol monomethyl ether, dipropylene glycol monomethyl ether, ethylene glycol monoethyl ether and diethylene glycol monoethyl ether, C₃-C₆ alkanediols, e.g. propane-1,2-diol, propane-1,3-diol and butane-1,2-diol, imidazolidin-2-one, and other compounds such as fatty alcohols, lanolin derivatives, active ingredients like pantothenic acid, agents for preventing hair loss, antidandruff agents, thickeners, suspending agents, sequestering agents, opacifying agents, colourants, sun filters, perfumes and preservatives.

The reducing composition for the hair according to the invention is presented essentially in aqueous form, especially in the form of a thickened or non-thickened lotion, a cream or a gel.

The reducing composition for the hair according to the invention can also be of the exothermic type, i.e. of the type causing a certain amount of warmth when it is applied to the hair, which is pleasant for the person undergoing the first step of the perming or uncurling procedure.

The reducing composition for the hair according to the invention can also contain a solvent, e.g. ethanol, propanol, isopropanol or glycerol, at a maximum concentration of 20%, based on the total weight of the composition.

The cosmetically acceptable medium of the reducing compositions for the hair according to the invention is preferably water or an aqueous-alcoholic solution in which the alcohol is C₁-C₈, such as ethanol, isopropanol or butanol.

If the compositions are intended for a hair uncurling or straightening operation, the reducing composition is preferably in the form of a thickened cream so as to keep the hair as stiff as possible. These creams are produced in the form of “heavy” emulsions based e.g. on glyceryl stearate, glycol stearate, self-emulsifying waxes or fatty alcohols.

It is also possible to use liquids or gels containing thickeners, such as carboxyvinylic polymers or copolymers, which “stick” the hair and keep it in the smooth position during the exposure time.

The invention further relates to a kit, especially for hair perming, comprising, in a first compartment, a composition according to the invention comprising a compound of formula (I) as reducing composition, and, in a second compartment, an oxidizing composition.

The present invention further relates, by way of novel compounds, to the aminodithiols of general formula (II) below:

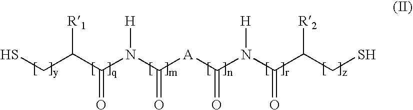

in which:

m, n, q, r=0 or 1 with the following conditions:

a) if m=n=0 and q=r=1,
(i) R′₁, and R′₂ independently of one another are CONR′₆R′₇, where R′₆, R′₇=H or linear or branched C₁-C₅ alkyl optionally substituted by 1 or 2 radicals selected from hydroxyl, methylamino, ethylamino and dimethylamino, y and z are equal to 1 or 2 and A is (CH₂)ₓ, CH₂—CHOH—CH₂ or CH₂—CHOH—CHOH—CH₂, x being equal to 1, 2, 3, 4, 5 or 6. If x=0, R′₁ and R′₂ can also be a hydrogen atom.
(ii) R′₁, and R′₂ are also a hydrogen atom when x is equal to 3, 4, 5 or 6 and y and z are equal to 2.

b) if m=n=0 and q=r=0,
(i) R′₁ and R′₂ independently of one another are CONR′₆R′₇, where R′₆, R′₇=H or linear or branched C₁-C₅ alkyl optionally substituted by 1 or 2 radicals selected from hydroxyl, methylamino, ethylamino and dimethylamino, y and z are equal to 1 or 2 and A is (CH₂)ₓ, CH₂—CHOH—CH₂ or CH₂—CHOH—CHOH—CH₂, x being equal to 1, 2, 3, 4, 5 or 6. If x=0, R′₁ and R′₂ can also be a hydrogen atom.
(ii) R′₁ and R′₂ are also a hydrogen atom when x is equal to 3, 4, 5 or 6 and y and z are equal to 2.
(iii) R′₁ and R′₂ are also a hydrogen atom when x is equal to 4, 5 or 6 and y and z are equal to 1.
   and their physiologically acceptable organic and inorganic salts.

The preferred compounds below may be mentioned in particular among the compounds of general formula (II):
2-[(1-carboxy-2-mercaptoethylaminooxalyl)amino]-3-mercaptopropionic acid
N,N′-bis[2-(methylamino)-2-oxo-1-(mercaptomethyl)ethyl]ethanediamide
N,N′-bis[2-(ethylamino)-2-oxo-1-(mercaptomethyl)ethyl]ethanediamide
N,N′-bis[2-(dimethylamino)-2-oxo-1-(mercaptomethyl)ethyl]ethanediamide
N,N′-bis[2-[2-hydroxypropylamino]-2-oxo-1-(mercaptomethyl)ethyl]ethanediamide
N,N′-bis[2-[2-hydroxyethylamino]-2-oxo-1-(mercaptomethyl)ethyl]ethanediamide
N,N′-bis[2-{[2-(dimethylamino)ethyl]amino}-2-oxo-1-(mercaptomethyl)ethyl]ethanediamide
N,N′-bis[2-{[3-(dimethylamino)propyl]amino}-2-oxo-1-(mercaptomethyl)ethyl]ethanediamide
2-({3-[(1-carboxy-2-mercaptoethyl)amino]-3-oxopropanoxyl}amino)-3-mercaptopropionic acid
N,N′-bis[2-(methylamino)-2-oxo-1-(mercaptomethyl)ethyl]malonamide
N,N′-bis[2-(ethylamino)-2-oxo-1-(mercaptomethyl)ethyl]malonamide
N,N′-bis[2-(2-hydroxypropylamino)-2-oxo-1-(mercaptomethyl)ethyl]malonamide
N,N′-bis[2-(2-hydroxyethylamino)-2-oxo-1-(mercaptomethyl)ethyl]malonamide
N,N′-bis[2-(dimethylamino)-2-oxo-1-(mercaptomethyl)ethyl]malonamide
N,N′-bis[2-{[2-(dimethylamino)ethyl]amino}-2-oxo-1-(mercaptomethyl)ethyl]malonamide
N,N′-bis[2-{[3-(dimethylamino)propyl]amino}-2-oxo-1-(mercaptomethyl)ethyl]malonamide
2-({[(1-carboxy-2-mercaptoethyl)amino]acetyl}amino)-3-mercaptopropionic acid
N-methyl-2-[({[2-methylamino-2-oxo-1-(mercaptomethyl)ethyl]amino}acetyl)amino]-3-mercaptomethylpropanamide
N-methyl-2-[({[2-hydroxypropylamino-2-oxo-1-(mercaptomethyl)ethyl]amino}acetyl)amino]-3-mercaptomethylpropanamide
N-methyl-2-[({[2-hydroxyethylamino-2-oxo-1-(mercaptomethyl)ethyl]amino}acetyl)amino]-3-mercaptomethylpropanamide
2-({[2-dimethylamino-2-oxo-1-(mercaptomethyl)ethyl]amino}acetyl)amino-N,N-dimethyl-3-mercaptopropanamide
2-({[2-diethylamino-2-oxo-1-(mercaptomethyl)ethyl]amino}acetyl)amino-N,N-dimethyl-3-mercaptopropanamide
N-[2-(dimethylamino)ethyl]-2-methyl-6,9-dioxo-7,12-bis(mercaptomethyl)-2,5,8,11-tetraazamidecan-13-amide
N-[3-(dimethylamino)propyl]-2-methyl-6,9-dioxo-7,12-bis(mercaptomethyl)-2,5,8,11-tetraazamidecan-13-amide
N,N′-(1,3-propanediyl)-bis-L-homocysteine
N,N′-1,3-(2-hydroxypropanediyl)-bis-L-cysteine
N,N′-1,3-(2-hydroxypropanediyl)-bis-L-homocysteine
N,N′-1,5-pentanediylbis(3-mercaptopropionamide)
N,N′-1,3-propanediylbis(4-mercaptobutanamide)
N,N′-1,3-(2-hydroxypropanediyl)bis(3-mercaptopropionamide)
N,N′-1,3-(2-hydroxypropanediyl)bis(4-mercaptobutanamide)
N,N′-1,4-butanediylbis(4-mercaptobutanamide)
N-[2-(2-carboxy-3-mercaptopropionylamino)ethyl]-2-mercaptomethylmalonamic acid
2-[2-(2-carbamoyl-3-mercaptopropionylamino)ethylcarbamoyl]-3-mercaptopropionamide
3-mercapto-N-[2-(3-mercapto-2-methylcarbamoylpropionylamino)ethyl]-2-methylcarbamoylpropionamide
3-mercapto-N-[2-(3-mercapto-2-ethylcarbamoylpropionylamino)ethyl]-2-ethylcarbamoylpropionamide
2-dimethylcarbamoyl-N-[2-(2-dimethylcarbamoyl-3-mercaptopropionylamino)ethyl]-3-mercaptopropionamide
N-[3-(2-carboxy-3-mercaptopropionylamino)propyl]-2-mercaptomethylmalonamic acid
2-[3-(2-carbamoyl-3-mercaptopropionylamino)propylcarbamoyl]-3-mercaptopropionamide
3-mercapto-N-[3-(3-mercapto-2-methylcarbamoylpropionylamino)propyl]-2-methylcarbamoylpropionamide
3-mercapto-N-[3-(3-mercapto-2-ethylcarbamoylpropionylamino)propyl]-2-ethylcarbamoylpropionamide
2-dimethylcarbamoyl-N-[3-(2-dimethylcarbamoyl-3-mercaptopropionylamino)propyl]-3-mercaptopropionamide
N-[3-(2-carboxy-3-mercaptopropionylamino)-2-hydroxypropyl]-2-mercaptomethylmalonamic acid
2-[3-(2-carbamoyl-3-mercaptopropionylamino)-2-hydroxypropylcarbamoyl]-3-mercaptopropionamide
3-mercapto-N-[3-(3-mercapto-2-methylcarbamoylpropionylamino)-2-hydroxypropyl]-2-methylcarbamoylpropionamide
3-mercapto-N-[3-(3-mercapto-2-ethylcarbamoylpropionylamino)-2-hydroxypropyl]-2-ethylcarbamoylpropionamide
2-dimethylcarbamoyl-N-[3-(2-dimethylcarbamoyl-3-mercaptopropionylamino)-2-hydroxypropyl]-3-mercaptopropionamide.

The following compounds are very particularly preferred:
2-[(1-carboxy-2-mercaptoethylaminooxalyl)amino]-3-mercaptopropionic acid
N,N′-bis[2-(methylamino)-2-oxo-1-(mercaptomethyl)ethyl]ethanediamide
N,N′-bis[2-(ethylamino)-2-oxo-1-(mercaptomethyl)ethyl]ethanediamide
N,N′-bis[2-(dimethylamino)-2-oxo-1-(mercaptomethyl)ethyl]ethanediamide
N,N′-bis[2-[2-hydroxypropylamino]-2-oxo-1-(mercaptomethyl)ethyl]ethanediamide
N,N′-bis[2-[2-hydroxyethylamino]-2-oxo-1-(mercaptomethyl)ethyl]ethanediamide
2-({3-[(1-carboxy-2-mercaptoethyl)amino]-3-oxopropanoxyl}amino)-3-mercaptopropionic acid
N,N′-bis[2-(methylamino)-2-oxo-1-(mercaptomethyl)ethyl]malonamide
N,N′-bis[2-(ethylamino)-2-oxo-1-(mercaptomethyl)ethyl]malonamide
N,N′-bis[2-(2-hydroxypropylamino)-2-oxo-1-(mercaptomethyl)ethyl]malonamide
N,N′-bis[2-(2-hydroxyethylamino)-2-oxo-1-(mercaptomethyl)ethyl]malonamide
N,N′-bis[2-(dimethylamino)-2-oxo-1-(mercaptomethyl)ethyl]malonamide
2-({[(1-carboxy-2-mercaptoethyl)amino]acetyl}amino)-3-mercaptopropionic acid
N-methyl-2-[({[2-methylamino-2-oxo-1-(mercaptomethyl)ethyl]amino}acetyl)amino]-3-mercaptomethylpropanamide
N-methyl-2-[({[2-hydroxypropylamino-2-oxo-1-(mercaptomethyl)ethyl]amino}acetyl)amino]-3-mercaptomethylpropanamide
N-methyl-2-[({[2-hydroxyethylamino-2-oxo-1-(mercaptomethyl)ethyl]amino}acetyl)amino]-3-mercaptomethylpropanamide
2-({[2-dimethylamino-2-oxo-1-(mercaptomethyl)ethyl]amino}acetyl)amino-N,N-dimethyl-3-mercaptopropanamide
N,N′-(1,3-propanediyl)-bis-L-homocysteine
N,N′-1,3-(2-hydroxypropanediyl)-bis-L-cysteine
N,N′-1,3-(2-hydroxypropanediyl)-bis-L-homocysteine
N,N′-1,3-propanediylbis(4-mercaptobutanamide)
N,N′-1,3-(2-hydroxypropanediyl)bis(3-mercaptopropionamide)
N,N′-1,3-(2-hydroxypropanediyl)bis(4-mercaptobutanamide)
N,N′-1,4-butanediylbis(4-mercaptobutanamide)
N-[2-(2-carboxy-3-mercaptopropionylamino)ethyl]-2-mercaptomethylmalonamic acid
2-[2-(2-carbamoyl-3-mercaptopropionylamino)ethylcarbamoyl]-3-mercaptopropionamide
3-mercapto-N-[2-(3-mercapto-2-methylcarbamoylpropionylamino)ethyl]-2-methylcarbamoylpropionamide
2-dimethylcarbamoyl-N-[2-(2-dimethylcarbamoyl-3-mercaptopropionylamino)ethyl]-3-mercaptopropionamide
N-[3-(2-carboxy-3-mercaptopropionylamino)propyl]-2-mercaptomethylmalonamic acid
2-[3-(2-carbamoyl-3-mercaptopropionylamino)propylcarbamoyl]-3-mercaptopropionamide
3-mercapto-N-[3-(3-mercapto-2-methylcarbamoylpropionylamino)propyl]-2-methylcarbamoylpropionamide
2-dimethylcarbamoyl-N-[3-(2-dimethylcarbamoyl-3-mercaptopropionylamino)propyl]-3-mercaptopropionamide
N-[3-(2-carboxy-3-mercaptopropionylamino)-2-hydroxypropyl]-2-mercaptomethylmalonamic acid
2-[3-(2-carbamoyl-3-mercaptopropionylamino)-2-hydroxypropylcarbamoyl]-3-mercaptopropionamide
3-mercapto-N-[3-(3-mercapto-2-methylcarbamoylpropionylamino)-2-hydroxypropyl]-2-methylcarbamoylpropionamide
2-dimethylcarbamoyl-N-[3-(2-dimethylcarbamoyl-3-mercaptopropionylamino)-2-hydroxypropyl]-3-mercaptopropionamide.

The compounds of formula (II) are prepared by processes known to those skilled in the art. The bibliographic references cited for the preparation of the compounds (I) are also applicable to the compounds of formula (II).

The invention will be understood more clearly with the aid of the following non-limiting Examples, which constitute preferred embodiments of the compositions according to the invention.

### PREPARATORY EXAMPLES

### Example 1

### Preparation of N,N′-1,3-(2-hydroxypropanediyl)bis(4-mercaptobutanamide)

The operations were performed under an inert atmosphere (argon).

4.5 g (0.05 mol) of 1,3-diamino-2-hydroxypropane were added all at once to 10.2 g (0.1 mol) of thiobutyrolactone.

The resulting solution was stirred for 1 h at room temperature and then for 1 h at a temperature of 40° C.-50° C. After the solution had been allowed to return to room temperature, 100 cm³ of acetone were added to give an attractive white precipitate. After filtration on a glass frit followed by drying under vacuum, 9.5 g of N,N′-1,3-(2-hydroxypropanediyl)bis(4-mercaptobutanamide) were obtained in the form of a white powder melting at 72.9° C.

The elemental analysis, the 400 MHz ¹H NMR and 100 MHz ¹³C NMR spectra and the mass spectrum are consistent with the expected structure.

The thiol titre determined by iodometry is 99.9%.

| Elemental analysis | C | H | N | O | S |
|---|---|---|---|---|---|
| Calculated | 44.90 | 7.50 | 9.50 | 16.30 | 21.80 |
| Found | 45.03 | 7.49 | 9.40 | 16.11 | 21.57 |

### COMPOSITION EXAMPLES

### Example 1

A reducing composition for hair perming was prepared according to the invention by mixing the following ingredients:

| | | |
|---|---|---|
| N,N′-1,3-(2-hydroxypropane- | 23.3 | g |
| diyl)bis(4-mercapto-butanamide) | | |
| 20% aqueous ammonia | qsp pH | 8.5 |
| demineralized water | qsp 100 | g |

This composition was applied to wet hair which had first been wound onto setting rollers. After the composition had been allowed to take effect for about 15 minutes, the hair with rollers was dried in a hair dryer for 5 minutes and then rinsed with copious amounts of water. The following oxidizing composition was then applied:

| | | |
|---|---|---|
| 200 volume hydrogen peroxide | 4.8 | g |
| citric acid | qsp pH | 3 |
| demineralized water | qsp 100 | g |

The oxidizing composition was allowed to take effect for 5 minutes and then rinsed off with copious amounts of water, the rollers were removed and the hair was dried under a hood. The attractive curls obtained were firm and lively.

### Example 2

A reducing composition for hair perming was prepared according to the invention by mixing the following ingredients:

| | | |
|---|---|---|
| N,N′-1,3-(2-hydroxypropane- | 14.5 | g |
| diyl)bis(4-mercapto-butanamide) | | |
| 20% aqueous ammonia | qsp pH | 8.5 |
| demineralized water | qsp 100 | g |

This composition was applied to wet hair which had first been wound onto setting rollers. After the composition had been allowed to take effect for about 25 minutes, the hair with rollers was dried in a hair dryer for 5 minutes and then rinsed with copious amounts of water. The following oxidizing composition was then applied:

| | | |
|---|---|---|
| 200 volume hydrogen peroxide | 4.8 | g |
| citric acid | qsp pH | 3 |
| demineralized water | qsp 100 | g |

The oxidizing composition was allowed to take effect for 5 minutes and then rinsed off with copious amounts of water, the rollers were removed and the hair was dried under a hood. The attractive curls obtained were firm and lively.

1. Use of at least one aminodithiol of general formula (I), in a reducing composition for the hair, as reducing agent for the first step of an operation for the perming of keratin fibres, especially hair: 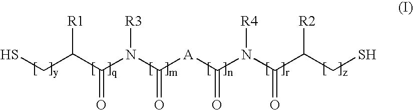
in which:
R₁ and R₂ independently of one another are H, COOR₅ or CONR₆R₇, where R₅=H or linear or branched C₁-C₅ alkyl optionally substituted by 1 or 2 OH radicals, and R₆, R₇=H or linear or branched C₁-C₅ alkyl optionally substituted by 1 or 2 radicals selected from hydroxyl, methylamino, ethylamino and dimethylamino,
R₃ and R₄ independently of one another are H or COOH,
y and z independently of one another are equal to 1 or 2,
A=nothing (0), (CH₂), where x ranges from 0 to 6, CH₂—CHOH—CH₂, CH₂—CHOH—CHOH—CH₂, HOOC—CH═CH—COOH or CH₂—CH(NR₈R₉)—(CH₂)ₚ, where p=1 or 2 and R₈, R₉=H, CH₃ or COCH₃,
m, n, q, r=0 or 1 with the following conditions:
(i) if m=n=1, q=r=0
(ii) if m=1 and n=0 and conversely if m=0 and n=1, q and r cannot simultaneously be equal to 1
(iii) if m=n=0, A cannot be nothing
(iv) m, n, q and r can simultaneously be equal to 0 if A is other than nothing,
and their physiologically acceptable organic and inorganic salts.

2. Use according to claim 1, characterized in that the compound of formula (I) is selected from:
2,2′-oxybis[N-(2-mercaptoethyl)acetamide]
N,N′-(1,2-dioxo-1,2-ethanediyl)bis(L-cysteine dimethyl ester)
N,N′-(1,3-dioxo-1,3-propanediyl)bis(L-cysteine dimethyl ester)
N,N′-(1,4-dioxo-1,4-butanediyl)bis(L-cysteine dimethyl ester)
N,N′-(1,2-dioxo-1,2-ethanediyl)-bis-L-cysteine
N,N′-(1,4-dioxo-1,4-butanediyl)-bis-L-cysteine
N,N′-(1,5-dioxo-1,5-pentanediyl)bis(L-cysteine dimethyl ester)
N,N′-(1,6-dioxo-1,6-hexanediyl)bis(L-cysteine dimethyl ester)
N,N′-(1,7-dioxo-1,7-heptanediyl)bis(L-cysteine dimethyl ester)
N,N′-(1,8-dioxo-1,8-octanediyl)bis(L-cysteine dimethyl ester)
N,N′-(1,2-ethanediyl)bis(L-cysteine dibutyl ester)
N,N′-(1,2-ethanediyl)bis(L-cysteine dimethyl ester)
N,N′-(1,2-ethanediyl)bis(L-cysteine dipropyl ester)
N,N′-(1,2-ethanediyl)-bis-L-cysteine
N,N′-(1,2-ethanediyl)bis(L-cysteine diethyl ester)
N,N′-(1,2-ethanediyl)bis(DL-cysteine diethyl ester)
N,N′-(1,2-ethanediyl)-bis-L-homocysteine
N,N′-(1,3-propanediyl)-bis-L-cysteine
N,N′-(1,3-propanediyl)-bis-L-homocysteine
N,N′-1,3-(2-hydroxypropanediyl)-bis-L-cysteine
N,N′-1,3-(2-hydroxypropanediyl)-bis-L-homocysteine
N,N′-carbonyl-bis(L-cysteine)
N-[2-[[(1R)-1-carboxy-2-mercaptoethyl]amino]ethyl]-L-cysteine 1-ethyl ester
N-[2-[(2-mercaptoethyl)amino]ethyl]-L-cysteine 1-ethyl ester
N-[2-[(2-mercaptoethyl)amino]ethyl]-L-cysteine
N-(2-mercaptoethyl)-2-[(2-mercaptoethyl)amino]-acetamide
N-[(2-mercaptoethyl)glycyl]-L-cysteine
N-[(2-mercaptoethyl)glycyl]-L-cysteine ethyl ester
N-(1-carboxy-2-mercaptoethyl)-3-[(1-carboxy-2-mercaptoethyl)amino]aspartic acid
2,2′-(ethylenediimino)diethanethiol dihydrochloride
3,7-diaza-1,9-nonanedithiol
3,6-diaza-1,8-octanedithiol
N,N′-bis(2-mercaptoethyl)ethanediamide
N,N′-bis(2-mercaptoethyl)butanediamide
N,N′-bis(2-mercaptoethyl)hexanediamide
1,4-bis[(2-mercaptoethyl)amino]-2,3-butanediol
N,N′-bis(mercaptoethyl)urea
1,2-ethanediylbis[(2-mercaptoethyl)carbamic] acid
2-[(1-carboxy-2-mercaptoethylaminooxalyl)amino]-3-mercaptopropionic acid
methyl 3-mercapto-2-[(2-mercapto-1-methoxycarbonylethylaminooxalyl)amino]propionate
ethyl 3-mercapto-2-[(2-mercapto-1-ethoxycarbonylethylaminooxalyl)amino]propionate
N,N′-bis[2-(methylamino)-2-oxo-1-(mercaptomethyl)ethyl]ethanediamide
N,N′-bis[2-(ethylamino)-2-oxo-1-(mercaptomethyl)ethyl]ethanediamide
N,N′-bis[2-(dimethylamino)-2-oxo-1-(mercaptomethyl)ethyl]ethanediamide
N,N′-bis[2-[(2-hydroxypropyl)amino]-2-oxo-1-(mercaptomethyl)ethyl]ethanediamide
N,N′-bis[2-[(2-hydroxyethyl)amino]-2-oxo-1-(mercaptomethyl)ethyl]ethanediamide
N,N′-bis[2-{[2-(dimethylamino)ethyl]amino}-2-oxo-1-(mercaptomethyl)ethyl]ethanediamide
N,N′-bis[2-{[3-(dimethylamino)propyl]amino}-2-oxo-1-(mercaptomethyl)ethyl]ethanediamide
2-({3-[(1-carboxy-2-mercaptoethyl)amino]-3-oxopropanoxyl}amino)-3-mercaptopropionic acid
methyl 2-[3-{[2-methoxy-2-oxo-1-(mercaptomethyl)ethyl]amino}-(3-oxopropanoxyl)amino]-3-mercaptopropanoate
ethyl 2-[3-{[2-ethoxy-2-oxo-1-(mercaptomethyl)ethyl]-amino}-(3-oxopropanoxyl)amino]-3-mercaptopropanoate
N,N′-bis[2-(methylamino)-2-oxo-1-(mercaptomethyl)ethyl]malonamide
N,N′-bis[2-(ethylamino)-2-oxo-1-(mercaptomethyl)ethyl]malonamide
N,N′-bis[2-(2-hydroxypropylamino)-2-oxo-1-(mercaptomethyl)ethyl]malonamide
N,N′-bis[2-(2-hydroxyethylamino)-2-oxo-1-(mercaptomethyl)ethyl]malonamide
N,N′-bis[2-(dimethylamino)-2-oxo-1-(mercaptomethyl)ethyl]malonamide
N,N′-bis[2-{[2-(dimethylamino)ethyl]amino}-2-oxo-1-(mercaptomethyl)ethyl]malonamide
N,N′-bis[2-{[3-(dimethylamino)propyl]amino}-2-oxo-1-(mercaptomethyl)ethyl]malonamide
2-({[(1-carboxy-2-mercaptoethyl)amino]acetyl}amino)-3-mercaptopropionic acid
methyl 2-[({[2-methoxy-2-oxo-1-(mercaptomethyl)ethyl]amino}acetyl)amino]-3-mercaptopropanoate
ethyl 2-[({[2-ethoxy-2-oxo-1-(mercaptomethyl)ethyl]amino}acetyl)amino]-3-mercaptopropanoate
N-methyl-2-[({[2-methylamino-2-oxo-1-(mercaptomethyl)ethyl]amino}acetyl)amino]-3-mercaptomethylpropanamide
N-methyl-2-[({[2-hydroxypropylamino-2-oxo-1-(mercaptomethyl)ethyl]amino}acetyl)amino]-3-mercaptomethylpropanamide
N-methyl-2-[({[2-hydroxyethylamino-2-oxo-1-(mercaptomethyl)ethyl]amino}acetyl)amino]-3-mercaptomethylpropanamide
2-({[2-dimethylamino-2-oxo-1-(mercaptomethyl)ethyl]amino}acetyl)amino-N,N-dimethyl-3-mercaptopropanamide
2-({[2-diethylamino-2-oxo-1-(mercaptomethyl)ethyl]amino}acetyl)amino-N,N-dimethyl-3-mercaptopropanamide
N-[2-(dimethylamino)ethyl]-2-methyl-6,9-dioxo-7,12-bis(mercaptomethyl)-2,5,8,11-tetraazamidecan-13-amide
N-[3-(dimethylamino)propyl]-2-methyl-6,9-dioxo-7,12-bis(mercaptomethyl)-2,5,8,11-tetraazamidecan-13-amide
N,N′-1,2-ethanediylbis(3-mercaptopropanamide)
N,N′-1,3-propanediylbis(3-mercaptopropanamide)
N,N′-1,3-(2-hydroxypropanediyl)bis(3-mercaptopropanamide)
N,N′-1,4-butanediylbis(3-mercaptopropanamide)
N,N′-1,5-pentanediylbis(3-mercaptopropanamide)
N,N′-1,6-hexanediylbis(3-mercaptopropanamide)
N,N′-1,2-ethanediylbis(4-mercaptobutanamide)
N,N′-1,3-propanediylbis(4-mercaptobutanamide)
N,N′-1,3-(2-hydroxypropanediyl)bis(4-mercaptobutanamide)
N,N′-1,4-butanediylbis(4-mercaptobutanamide)
N-[2-(2-carboxy-3-mercaptopropionylamino)ethyl]-2-mercaptomethylmalonamic acid
methyl N-[2-(2-methoxycarbonyl-3-mercaptopropionylamino)ethyl]-2-mercaptomethylmalonamate
ethyl N-[2-(2-ethoxycarbonyl-3-mercaptopropionylamino)ethyl]-2-mercaptomethylmalonamate
2-[2-(2-carbamoyl-3-mercaptopropionylamino)ethylcarbamoyl]-3-mercaptopropionamide
3-mercapto-N-[2-(3-mercapto-2-methylcarbamoylpropionylamino)ethyl]-2-methylcarbamoylpropionamide
3-mercapto-N-[2-(3-mercapto-2-ethylcarbamoylpropionylamino)ethyl]-2-ethylcarbamoylpropionamide
2-dimethylcarbamoyl-N-[2-(2-dimethylcarbamoyl-3-mercaptopropionylamino)ethyl]-3-mercaptopropionamide
N-[3-(2-carboxy-3-mercaptopropionylamino)propyl]-2-mercaptomethylmalonamic acid
methyl N-[3-(2-methoxycarbonyl-3-mercaptopropionylamino)propyl]-2-mercaptomethylmalonamate
ethyl N-[3-(2-ethoxycarbonyl-3-mercaptopropionylamino)propyl]-2-mercaptomethylmalonamate
2-[3-(2-carbamoyl-3-mercaptopropionylamino)propylcarbamoyl]-3-mercaptopropionamide
3-mercapto-N-[3-(3-mercapto-2-methylcarbamoylpropionylamino)propyl]-2-methylcarbamoylpropionamide
3-mercapto-N-[3-(3-mercapto-2-ethylcarbamoylpropionylamino)propyl]-2-ethylcarbamoylpropionamide
2-dimethylcarbamoyl-N-[3-(2-dimethylcarbamoyl-3-mercaptopropionylamino)propyl]-3-mercaptopropionamide
N-[3-(2-carboxy-3-mercaptopropionylamino)-2-hydroxypropyl]-2-mercaptomethylmalonamic acid
methyl N-[3-(2-methoxycarbonyl-3-mercaptopropionylamino)-2-hydroxypropyl]-2-mercaptomethylmalonamate
ethyl N-[3-(2-ethoxycarbonyl-3-mercaptopropionylamino)-2-hydroxypropyl]-2-mercaptomethylmalonamate
2-[3-(2-carbamoyl-3-mercaptopropionylamino)-2-hydroxypropylcarbamoyl]-3-mercaptopropionamide
3-mercapto-N-[3-(3-mercapto-2-methylcarbamoylpropionylamino)-2-hydroxypropyl]-2-methylcarbamoylpropionamide
3-mercapto-N-[3-(3-mercapto-2-ethylcarbamoylpropionylamino)-2-hydroxypropyl]-2-ethylcarbamoylpropionamide
2-dimethylcarbamoyl-N-[3-(2-dimethylcarbamoyl-3-mercaptopropionylamino)-2-hydroxypropyl]-3-mercaptopropionamide.

3. Use according to claim 1, characterized in that the compound of formula (I) is selected from:
2,2′-oxybis[N-(2-mercaptoethyl)acetamide]
N,N′-(1,2-dioxo-1,2-ethanediyl)bis(L-cysteine dimethyl ester)
N,N′-(1,4-dioxo-1,4-butanediyl)bis(L-cysteine dimethyl ester)
N,N′-(1,4-dioxo-1,4-butanediyl)-bis-L-cysteine
N,N′-(1,2-ethanediyl)-bis-L-cysteine
N,N′-(1,2-ethanediyl)-bis-L-homocysteine
N,N′-(1,3-propanediyl)-bis-L-cysteine
N,N′-(1,3-propanediyl)-bis-L-homocysteine
N,N′-1,3-(2-hydroxypropanediyl)-bis-L-cysteine
N,N′-1,3-(2-hydroxypropanediyl)-bis-L-homocysteine
N,N′-carbonylbis(L-cysteine)
N-[2-[[(1R)-1-carboxy-2-mercaptoethyl]amino]ethyl]-L-cysteine 1-ethyl ester
N-[2-[(2-mercaptoethyl)amino]ethyl]-L-cysteine 1-ethyl ester
N-[2-[(2-mercaptoethyl)amino]ethyl]-L-cysteine
N-(2-mercaptoethyl)-2-[(2-mercaptoethyl)amino]-acetamide
N-[(2-mercaptoethyl)glycyl]-L-cysteine
N-(1-carboxy-2-mercaptoethyl)-3-[(1-carboxy-2-mercaptoethyl)amino]aspartic acid
2,2′-(ethylenediimino)diethanethiol dihydrochloride
3,6-diaza-1,8-octanedithiol
N,N′-bis(2-mercaptoethyl)ethanediamide
N,N′-bis(2-mercaptoethyl)butanediamide
N,N′-bis(2-mercaptoethyl)hexanediamide
1,4-bis[(2-mercaptoethyl)amino]-2,3-butanediol
N,N′-bis(mercaptoethyl)urea
1,2-ethanediylbis[(2-mercaptoethyl)carbamic] acid
N,N′-1,2-ethanediylbis(3-mercaptopropanamide)
N,N′-1,3-propanediylbis(3-mercaptopropanamide)
N,N′-1,3-(2-hydroxypropanediyl)bis(3-mercaptopropanamide)
N,N′-1,4-butanediylbis(3-mercaptopropanamide)
N,N′-1,5-pentanediylbis(3-mercaptopropanamide)
N,N′-1,2-ethanediylbis(4-mercaptobutanamide)
N,N′-1,3-propanediylbis(4-mercaptobutanamide)
N,N′-1,3-(2-hydroxypropanediyl)bis(4-mercaptobutanamide)
N,N′-1,4-butanediylbis(4-mercaptobutanamide)
N-[2-(2-carboxy-3-mercaptopropionylamino)ethyl]-2-mercaptomethylmalonamic acid
methyl N-[2-(2-methoxycarbonyl-3-mercaptopropionylamino)ethyl]-2-mercaptomethylmalonamate
ethyl N-[2-(2-ethoxycarbonyl-3-mercaptopropionylamino)ethyl]-2-mercaptomethylmalonamate
2-[2-(2-carbamoyl-3-mercaptopropionylamino)ethylcarbamoyl]-3-mercaptopropionamide
3-mercapto-N-[2-(3-mercapto-2-methylcarbamoylpropionylamino)ethyl]-2-methylcarbamoylpropionamide
3-mercapto-N-[2-(3-mercapto-2-ethylcarbamoylpropionylamino)ethyl]-2-ethylcarbamoylpropionamide
2-dimethylcarbamoyl-N-[2-(2-dimethylcarbamoyl-3-mercaptopropionylamino)ethyl]-3-mercaptopropionamide
N-[3-(2-carboxy-3-mercaptopropionylamino)propyl]-2-mercaptomethylmalonamic acid
methyl N-[3-(2-methoxycarbonyl-3-mercaptopropionylamino)propyl]-2-mercaptomethylmalonamate
ethyl N-[3-(2-ethoxycarbonyl-3-mercaptopropionylamino)propyl]-2-mercaptomethylmalonamate
2-[3-(2-carbamoyl-3-mercaptopropionylamino)propylcarbamoyl]-3-mercaptopropionamide
3-mercapto-N-[3-(3-mercapto-2-methylcarbamoylpropionylamino)propyl]-2-methylcarbamoylpropionamide
3-mercapto-N-[3-(3-mercapto-2-ethylcarbamoylpropionylamino)propyl]-2-ethylcarbamoylpropionamide
2-dimethylcarbamoyl-N-[3-(2-dimethylcarbamoyl-3-mercaptopropionylamino)propyl]-3-mercaptopropionamide
N-[3-(2-carboxy-3-mercaptopropionylamino)-2-hydroxypropyl]-2-mercaptomethylmalonamic acid
methyl N-[3-(2-methoxycarbonyl-3-mercaptopropionylamino)-2-hydroxypropyl]-2-mercaptomethylmalonamate
ethyl N-[3-(2-ethoxycarbonyl-3-mercaptopropionylamino)-2-hydroxypropyl]-2-mercaptomethylmalonamate
2-[3-(2-carbamoyl-3-mercaptopropionylamino)-2-hydroxypropylcarbamoyl]-3-mercaptopropionamide
3-mercapto-N-[3-(3-mercapto-2-methylcarbamoylpropionylamino)-2-hydroxypropyl]-2-methylcarbamoylpropionamide
3-mercapto-N-[3-(3-mercapto-2-ethylcarbamoylpropionylamino)-2-hydroxypropyl]-2-ethylcarbamoylpropionamide
2-dimethylcarbamoyl-N-[3-(2-dimethylcarbamoyl-3-mercaptopropionylamino)-2-hydroxypropyl]-3-mercaptopropionamide.

4. Use according to claim 1, characterized in that the reducing agent of general formula (I) is advantageously present, in a cosmetically acceptable medium, at a concentration of between 0.05 and 35% by weight, preferably of between 1 and 25% by weight, based on the total weight of the reducing composition.

5. Use according to claims 1 to 4, characterized in that the pH of the reducing composition for the hair is between 4 and 11, more particularly between 6 and 10.

6. Use according to any one of the preceding claims, characterized in that the reducing composition for the hair also contains a surfactant of non-ionic, anionic, cationic or amphoteric type.

7. Use according to claim 6, characterized in that the surfactant is present at a maximum concentration of 30% by weight, preferably of between 0.5 and 10% by weight, based on the total weight of the reducing composition.

8. Use according to claim 4, characterized in that the cosmetically acceptable medium is water or an aqueous-alcoholic solution in which the alcohol is C₁-C₈, such as ethanol, isopropanol or butanol.

9. Hair perming method comprising the application of a reducing composition comprising, as reducing agent, a compound of formula (I): 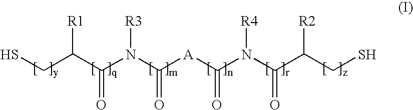
in which:
R₁ and R₂ independently of one another are H, COOR₅ or CONR₆R₇, where R₅=H or linear or branched C₁-C₅ alkyl optionally substituted by 1 or 2 OH radicals, and R₆, R₇=H or linear or branched C₁-C₅ alkyl optionally substituted by 1 or 2 radicals selected from hydroxyl, methylamino, ethylamino and dimethylamino,
R₃ and R₄ independently of one another are H or COOH,
y and z independently of one another are equal to 1 or 2,
A=nothing (0), (CH₂)ₓ, where x ranges from 0 to 6, CH₂—CHOH—CH₂, CH₂—CHOH—CHOH—CH₂, HOOC—CH═CH—COOH or CH₂—CH(NR₈R₉)—(CH₂)ₚ, where p=1 or 2 and R₈, R₉=H, CH₃ or COCH₃,
m, n, q, r=0 or 1 with the following conditions:
(i) if m=n=1, q=r=0
(ii) if m=1 and n=0 and conversely if m=0 and n=1, q and r cannot simultaneously be equal to 1
(iii) if m=n=0, A cannot be nothing
(iv) m, n, q and r can simultaneously be equal to 0 if A is other than nothing,
and their physiologically acceptable organic and inorganic salts.

10. Method according to claim 9, characterized in that the hair perming consists, in a first step, in reducing the disulphide bonds of the keratin by applying the reducing composition for about 5 to 60 min and then, in a second step, in re-forming said bonds by applying an oxidizing composition or optionally by allowing the atmospheric oxygen to take effect.

11. Method according to claim 9 or 10, characterized in that, after application of the reducing composition, the hair is subjected to a heat treatment by heating to a temperature of between 30 and 60° C.

12. Method according to claim 9, characterized in that the hair shaping consists of a hair uncurling or straightening method in which the reducing composition is applied to the hair, after which the hair is subjected to mechanical shaping in order to fix it in its new shape.

13. Hair perming kit comprising, in a first compartment, a composition according to the invention comprising a compound of formula (I) as reducing composition, and, in a second compartment, an oxidizing composition, 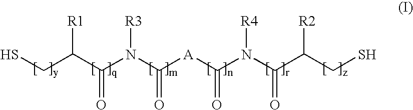
in which:
R₁ and R₂ independently of one another are H, COOR₅ or CONR₆, R₇, where R₅=H or linear or branched C₁-C₅ alkyl optionally substituted by 1 or 2 OH radicals, and R₆, R₇=H or linear or branched C₁-C₅ alkyl optionally substituted by 1 or 2 radicals selected from hydroxyl, methylamino, ethylamino and dimethylamino,
R₃ and R₄ independently of one another are H or COOH,
y and z independently of one another are equal to 1 or 2,
A=nothing (0), (CH₂)ₓ, where x ranges from 0 to 6, CH₂—CHOH—CH₂, CH₂—CHOH—CHOH—CH₂, HOOC—CH═CH—COOH or CH₂—CH(NR₈R₉)—(CH₂)ₚ, where p=1 or 2 and R₈, R₉=H, CH₃ or COCH₃,
m, n, q, r=0 or 1 with the following conditions:
(i) if m=n=1, q=r=0
(ii) if m=1 and n=0 and conversely if m=0 and n=1, q and r cannot simultaneously be equal to 1
(iii) if m=n=0, A cannot be nothing
(iv) m, n, q and r can simultaneously be equal to 0 if A is other than nothing,
and their physiologically acceptable organic and inorganic salts.

14. Aminodithiols of general formula (II) below: 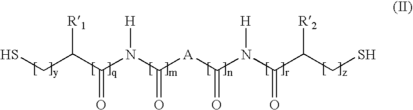
in which:
m, n, q, r=0 or 1 with the following conditions:
a) if m=n=0 and q=r=1,
(i) R′₁ and R′₂ independently of one another are CONR′₆R′₇, where R′₆, R′₇=H or linear or branched C₁-C₅ alkyl optionally substituted by 1 or 2 radicals selected from hydroxyl, methylamino, ethylamino and dimethylamino, y and z are equal to 1 or 2 and A is (CH₂)ₓ, CH₂—CHOH—CH₂ or CH₂—CHOH—CHOH—CH₂, x being equal to 1, 2, 3, 4, 5 or 6. If x=0, R′₁ and R′₂ can also be a hydrogen atom.
(ii) R′₁ and R′₂ are also a hydrogen atom when x is equal to 3, 4, 5 or 6 and y and z are equal to 2.
b) if m=n=0 and q=r=0,
(i) R′₁ and R′₂ independently of one another are CONR′₆R′₇, where R′₆, R′₇=H or linear or branched C₁-C₅ alkyl optionally substituted by 1 or 2 radicals selected from hydroxyl, methylamino, ethylamino and dimethylamino, y and z are equal to 1 or 2 and A is (CH₂)ₓ, CH₂—CHOH—CH₂ or CH₂—CHOH—CHOH—CH₂, x being equal to 1, 2, 3, 4, 5 or 6. If x=0, R′₁ and R′₂ can also be a hydrogen atom.
(ii) R′₁ and R′₂ are also a hydrogen atom when x is equal to 3, 4, 5 or 6 and y and z are equal to 2.
(iii) R′₁ and R′₂ are also a hydrogen atom when x is equal to 4, 5 or 6 and y and z are equal to 1.
and their physiologically acceptable organic and inorganic salts.

15. Aminodithiol of formula (II) according to claim 14, selected from:
2-[(1-carboxy-2-mercaptoethylaminooxalyl)amino]-3-mercaptopropionic acid
N,N′-bis[2-(methylamino)-2-oxo-1-(mercaptomethyl)ethyl]ethanediamide
N,N′-bis[2-(ethylamino)-2-oxo-1-(mercaptomethyl)ethyl]ethanediamide
N,N′-bis[2-(dimethylamino)-2-oxo-1-(mercaptomethyl)ethyl]ethanediamide
N,N′-bis[2-[2-hydroxypropylamino]-2-oxo-1-(mercaptomethyl)ethyl]ethanediamide
N,N′-bis[2-[2-hydroxyethylamino]-2-oxo-1-(mercaptomethyl)ethyl]ethanediamide
N,N′-bis[2-{[2-(dimethylamino)ethyl]amino}-2-oxo-1-(mercaptomethyl)ethyl]ethanediamide
N,N′-bis[2-{[3-(dimethylamino)propyl]amino}-2-oxo-1-(mercaptomethyl)ethyl]ethanediamide
2-({3-[(1-carboxy-2-mercaptoethyl)amino]-3-oxopropanoxyl}amino)-3-mercaptopropionic acid
N,N′-bis[2-(methylamino)-2-oxo-1-(mercaptomethyl)ethyl]malonamide
N,N′-bis[2-(ethylamino)-2-oxo-1-(mercaptomethyl)ethyl]malonamide
N,N′-bis[2-(2-hydroxypropylamino)-2-oxo-1-(mercaptomethyl)ethyl]malonamide
N,N′-bis[2-(2-hydroxyethylamino)-2-oxo-1-(mercaptomethyl)ethyl]malonamide
N,N′-bis[2-(dimethylamino)-2-oxo-1-(mercaptomethyl)ethyl]malonamide
N,N′-bis[2-{[2-(dimethylamino)ethyl]amino}-2-oxo-1-(mercaptomethyl)ethyl]malonamide
N,N′-bis[2-{[3-(dimethylamino)propyl]amino}-2-oxo-1-(mercaptomethyl)ethyl]malonamide
2-({[(1-carboxy-2-mercaptoethyl)amino]acetyl}amino)-3-mercaptopropionic acid
N-methyl-2-[({[2-methylamino-2-oxo-1-(mercaptomethyl)ethyl]amino}acetyl)amino]-3-mercaptomethylpropanamide
N-methyl-2-[({[2-hydroxypropylamino-2-oxo-1-(mercaptomethyl)ethyl]amino}acetyl)amino]-3-mercaptomethylpropanamide
N-methyl-2-[({[2-hydroxyethylamino-2-oxo-1-(mercaptomethyl)ethyl]amino}acetyl)amino]-3-mercaptomethylpropanamide
2-({[2-dimethylamino-2-oxo-1-(mercaptomethyl)ethyl]amino}acetyl)amino-N,N-dimethyl-3-mercaptopropanamide
2-({[2-diethylamino-2-oxo-1-(mercaptomethyl)ethyl]amino}acetyl)amino-N,N-dimethyl-3-mercaptopropanamide
N-[2-(dimethylamino)ethyl]-2-methyl-6,9-dioxo-7,12-bis(mercaptomethyl)-2,5,8,11-tetraazamidecan-13-amide
N-[3-(dimethylamino)propyl]-2-methyl-6,9-dioxo-7,12-bis(mercaptomethyl)-2,5,8,11-tetraazamidecan-13-amide
N,N′-(1,3-propanediyl)-bis-L-homocysteine
N,N′-1,3-(2-hydroxypropanediyl)-bis-L-cysteine
N,N′-1,3-(2-hydroxypropanediyl)-bis-L-homocysteine
N,N′-1,5-pentanediylbis(3-mercaptopropionamide)
N,N′-1,3-propanediylbis(4-mercaptobutanamide)
N,N′-1,3-(2-hydroxypropanediyl)bis(3-mercaptopropionamide)
N,N′-1,3-(2-hydroxypropanediyl)bis(4-mercaptobutanamide)
N,N′-1,4-butanediylbis(4-mercaptobutanamide)
N-[2-(2-carboxy-3-mercaptopropionylamino)ethyl]-2-mercaptomethylmalonamic acid
2-[2-(2-carbamoyl-3-mercaptopropionylamino)ethylcarbamoyl]-3-mercaptopropionamide
3-mercapto-N-[2-(3-mercapto-2-methylcarbamoylpropionylamino)ethyl]-2-methylcarbamoylpropionamide
3-mercapto-N-[2-(3-mercapto-2-ethylcarbamoylpropionylamino)ethyl]-2-ethylcarbamoylpropionamide
2-dimethylcarbamoyl-N-[2-(2-dimethylcarbamoyl-3-mercaptopropionylamino)ethyl]-3-mercaptopropionamide
N-[3-(2-carboxy-3-mercaptopropionylamino)propyl]-2-mercaptomethylmalonamic acid
2-[3-(2-carbamoyl-3-mercaptopropionylamino)propylcarbamoyl]-3-mercaptopropionamide
3-mercapto-N-[3-(3-mercapto-2-methylcarbamoylpropionylamino)propyl]-2-methylcarbamoylpropionamide
3-mercapto-N-[3-(3-mercapto-2-ethylcarbamoylpropionylamino)propyl]-2-ethylcarbamoylpropionamide
2-dimethylcarbamoyl-N-[3-(2-dimethylcarbamoyl-3-mercaptopropionylamino)propyl]-3-mercaptopropionamide
N-[3-(2-carboxy-3-mercaptopropionylamino)-2-hydroxypropyl]-2-mercaptomethylmalonamic acid
2-[3-(2-carbamoyl-3-mercaptopropionylamino)-2-hydroxypropylcarbamoyl]-3-mercaptopropionamide
3-mercapto-N-[3-(3-mercapto-2-methylcarbamoylpropionylamino)-2-hydroxypropyl]-2-methylcarbamoylpropionamide
3-mercapto-N-[3-(3-mercapto-2-ethylcarbamoylpropionylamino)-2-hydroxypropyl]-2-ethylcarbamoylpropionamide
2-dimethylcarbamoyl-N-[3-(2-dimethylcarbamoyl-3-mercaptopropionylamino)-2-hydroxypropyl]-3-mercaptopropionamide.
